(19) United States
(12) Patent Application Publication
(10) Pub. No.: US 2016/0183826 A1
(43) Pub. Date: Jun. 30, 2016

(54) SYSTEM AND METHOD OF SERIAL COMPARISON OF 12-LEAD ELECTROCARDIOGRAM (ECG) DURING EPISODE-OF-CARE

(71) Applicant: General Electric Company, Schenectady, NY (US)

(72) Inventors: Gordon Ian Rowlandson, Wauwatosa, WI (US); Joel Qiuzhen Xue, Wauwatosa, WI (US); Brian J. Young, Wauwatosa, WI (US); Anthony Holmes, Wauwatosa, WI (US)

(73) Assignee: GENERAL ELECTRIC COMPANY, Schenectady, NY (US)

(21) Appl. No.: 14/587,584

(22) Filed: Dec. 31, 2014

(51) Int. Cl. A61B 5/04; A61B 5/00; A61B 5/0452

(52) U.S. Cl. CPC A61B 5/04012; A61B 5/0452; A61B 5/742; A61B 5/04011

(57) ABSTRACT

The system and method of the present application selects and presents ECGs that are most important to the user in conjunction with a measurement trend that relates to the diagnosis and management of the abnormality. In addition, the system and method of the present application will guide the user to verify whether the ECGs selected by the computer were valid and if not guide the user through measurement trends to find 12-ECGs of significance.

### FIELD

The present disclosure generally relates to diagnostic cardiology. More specifically, the present disclosure relates to serial electrocardiogram (ECG) comparison.

### BACKGROUND

In electrocardiography, the ST segment connects the QRS complex and the wave.

It starts at the J point (junction between the QRS complex and ST segment) and ends at the beginning of the T wave. However, since it is usually difficult to determine exactly where the ST segment ends and the T wave begins, the relationship between the ST segment and T wave should be examined together. Serial comparison of multiple 12-lead ECGs from a single patient is considered a standard of care during certain episodes-of-care, such as the detection and management of an acute coronary syndrome (ACS). Manual methods for doing this are time consuming. Furthermore, there can he so many 12-lead ECGs to be reviewed that critical changes can be overlooked.

The serial ECG is instrumental for solving particular problems, such as the accurate identification of patients with ACS. However, oftentimes there is too much information for the clinician to handle. In current systems, information is assessed and presented in a succinct manner, that is, the 12-lead ECGs that are relevant to the decision along with an ST segment trend (ST trend) of a measurement that pertains to the abnormality. However, it should also be clear that trends have their own problems, as set forth below. What is needed is a tool to allow a user to more efficiently and effectively navigate ST trends for the clinically relevant information.

ST trends have many problems. First, ST trends can be corrupted with artifact. Without the user able to look at the raw data, it is hard to determine whether a change in an ST segment is indeed artifact. Also, to effectively use an ST trend, one needs to select which lead is most impacted by a change. Also, some use ST vector magnitude as a method to identify critical episodes. However, the direction of the ST vector is often more important than the magnitude. One also needs to know if an ST change is due to depolarization, and further needs to rule out secondary repolarization abnormality. Furthermore, typically users react to the 12-lead ECG, not a trend, and serial comparison programs are not able to automatically select the best example of the change and show the ECGs related to this change.

Current serial comparison programs were never optimized for the purposes of evaluating a patient during an episode-of-care for a specific condition such as the evaluation of a patient suspected of an ACS or due to the administration of a drug known to prolong QT and cause sudden cardiac death. Rather, the existing serial comparison program mimicked the activity that occurs in the typical heart station where incoming ECGs that need to be edited are routinely compared to a prior ECG. This is in contrast to an episode-of-care where many ECGs are acquired on a single patient as often as every 30 minutes over several days in accordance with published guidelines.

### SUMMARY

The system and method of the present application selects and presents ECGs that are most important to the user in conjunction with a measurement trend that relates to the diagnosis and management of the abnormality. In addition, the system and method of the present application will guide the user to verify whether the ECGs selected by the computer were valid and, if not, guide the user through measurement trends to find 12-ECGs of significance

In one aspect of the present application, a method of serial comparison of a plurality of 12-lead electrocardiograms ECGs) during an episode of care, comprises determining, a vector for each of the plurality of 12-lead ECGs, wherein each vector represents energy for a time segment, generating a trend for each vector, calculating a signal quality for a set of candidate ECGs selected from the plurality of 12-lead ECGs, and determining an ECG from the set of candidate ECGs that best reflects a change in the trend.

In another aspect of the present application, a system of serial comparison of a plurality of 12-lead electrocardiograms (ECGs) during an episode of care, comprises a controller configured to determine a vector for each of the plurality of 12-lead ECGs, wherein each vector represents energy for a time segment, and generate a trend for each vector, the controller further configured to calculate a signal quality for a set of candidate ECGs selected from the plurality of 12-lead ECGs, and to determine an ECG from the set of candidate ECGs that best reflects a change in the trend, and a graphical user interface configured to display the trend using a lead selected that reflects a greatest change.

In another aspect of the present application, a method of serial comparison of a plurality of 12-lead electrocardiograms (ECGs) during an episode of care, comprises synthesizing a set of XYZ components for each of the plurality of 12-lead ECGs, computing a representative complex for each of the plurality of 12-lead ECGs and the set of XYZ component, integrating a voltage for each of the X, Y and Z components, and further calculating a vector magnitude and a spatial angle for each vector magnitude, generating a trend for each vector magnitude, calculating a signal quality for a set of candidate ECGs selected from the plurality of 12-lead ECGs, determining an ECG from the set of candidate ECGs that best reflects a change in the trend, and rendering the trend using a lead selected that reflects a greatest change.

### BRIEF DESCRIPTION OF THE DRAWINGS

FIG. 1 is a flowchart of a method illustrating an embodiment of the present application;

FIG. 2 is a flowchart of a method illustrating an embodiment of the present application;

FIG. 3 is a flowchart of a method illustrating an embodiment of the present application;

FIG. 4 is a view illustrating a graphical user interface (GUI) of an embodiment of the present application;

FIG. 5 is a view illustrating a graphical user interface (GUI) of an embodiment of the present application;

FIG. 6 is a flowchart illustrating a method illustrating an embodiment of the present application;

FIG. 7 is a view illustrating a graphical user interface (GUI) of an embodiment of the present application; and

FIG. 8 is a schematic diagram of a system illustrating an embodiment of the e present application.

### DETAILED DESCRIPTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and methods described herein may be used alone or in combination with other systems and methods. Various equivalents, alternatives and modifications are possible within the scope of the appended claims. Each limitation in the appended claims is intended to invoke interpretation under 35 U.S.C. §112, sixth paragraph, only if the terms “means for” or “step for” are explicitly recited in the respective limitation.

In the following detailed description, reference is made to the accompanying drawings that form a part hereof, and in which is shown by way of illustration specific embodiments that may he practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

The technical effect of the system and method of the present application is to improve the function and operation of current ECG monitor devices and ECG serial comparison systems and methods to include more detailed and useful information for a user.

In this system and method, a graphical user display and interface (GUI) is used for both projecting, what the system and method sets as the best view of the episode of care as well as for the user to navigate through this information to either confirm or disregard the conclusions made by the system and method.

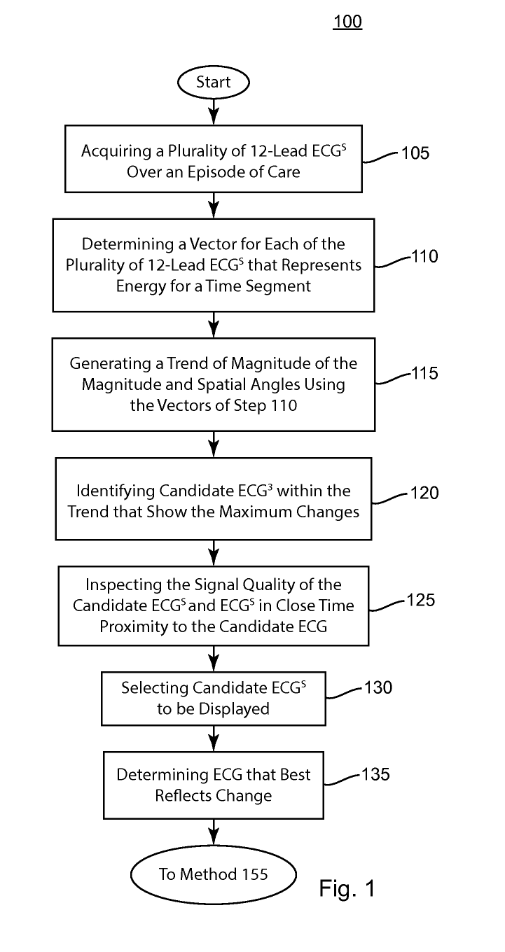

In order to create this GUI, the system and method performs the following steps set forth in FIG. 1. First, in step 105, the system acquires a plurality of 12-lead ECGs over an episode of care, and for each 12-lead ECG acquired over the episode of care in step 105, determining in step 110 for any section of the cardiac complex, a vector that represents the energy for that segment. This step is not limited, to the ST segment and this can be done for such defined, waves of the cardiac complex as the Q-wave, QRS, ST segment or T-wave or any arbitrarily defined segment of time of the cardiac complex. In step 110, the vector is determined for a segment of time.

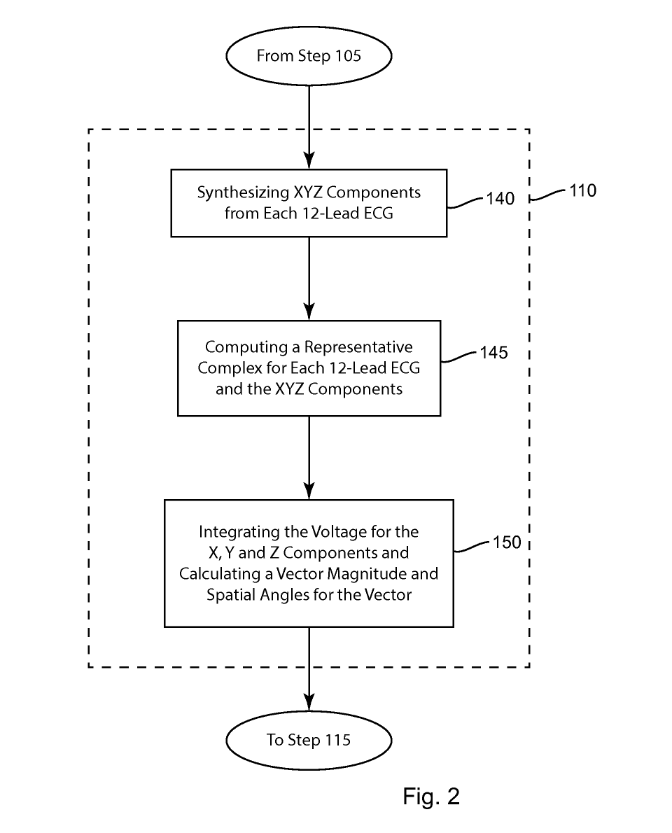

Referring, to FIG. 1 and FIG. 2, the step 110 is further carried out by synthesizing in step 140. XYZ components from each 12-lead ECG using various formulas known in the art, and computing in step 145 a representative complex for each lead of the 12-lead ECG plus the 3 synthesized XYZ components. In step 145, for the time segment defined, the voltage for the XYZ components is integrated. With these integrated voltages, a vector magnitude and spatial angles are calculated for the vector.

Referring again to FIG. 1, in step 115 the method 100 uses the computed vector values for each 12-lead ECG acquired in step 110 to generate a trend of the magnitude and spatial angles. In step 120, the method 100 identifies candidates within the trend that show the maximum changes either in terms of vector magnitude or angle, and in step 125, inspects the signal quality of these candidates as well as the neighboring ECGs of these candidates to insure that this is truly a physiological change. Typically, this signal quality is calculated with methods known in the art.

In step 130, the method 100 selects candidate ECGs to be displayed based on signal quality as well as maximum change, and in step 135, determines which lead of the 12-lead. ECG would best reflect the change that has been detected spatially. As an example only, if the maximum spatial change occurs anteriorly, select V2, or if inferiorly/superiorly, select II or laterally V5.

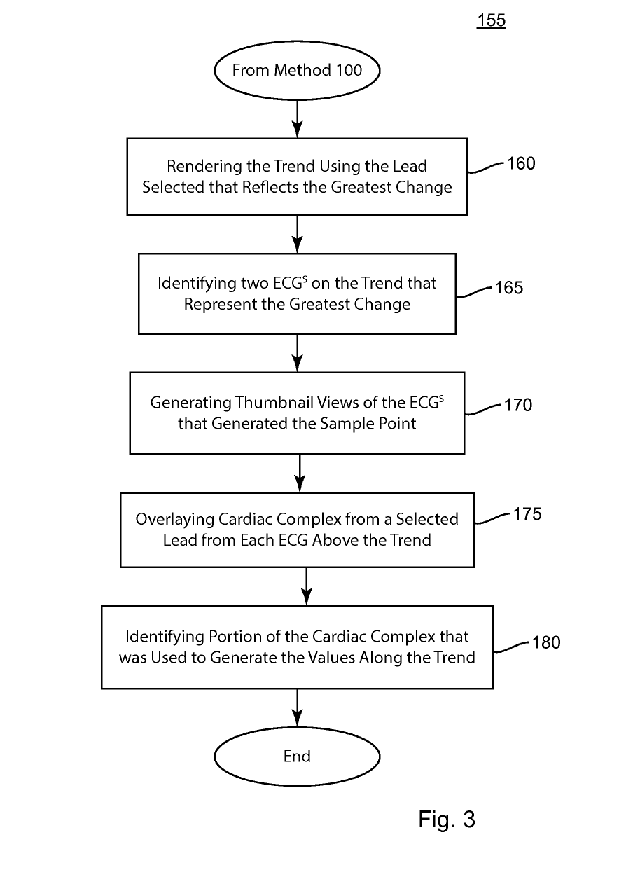

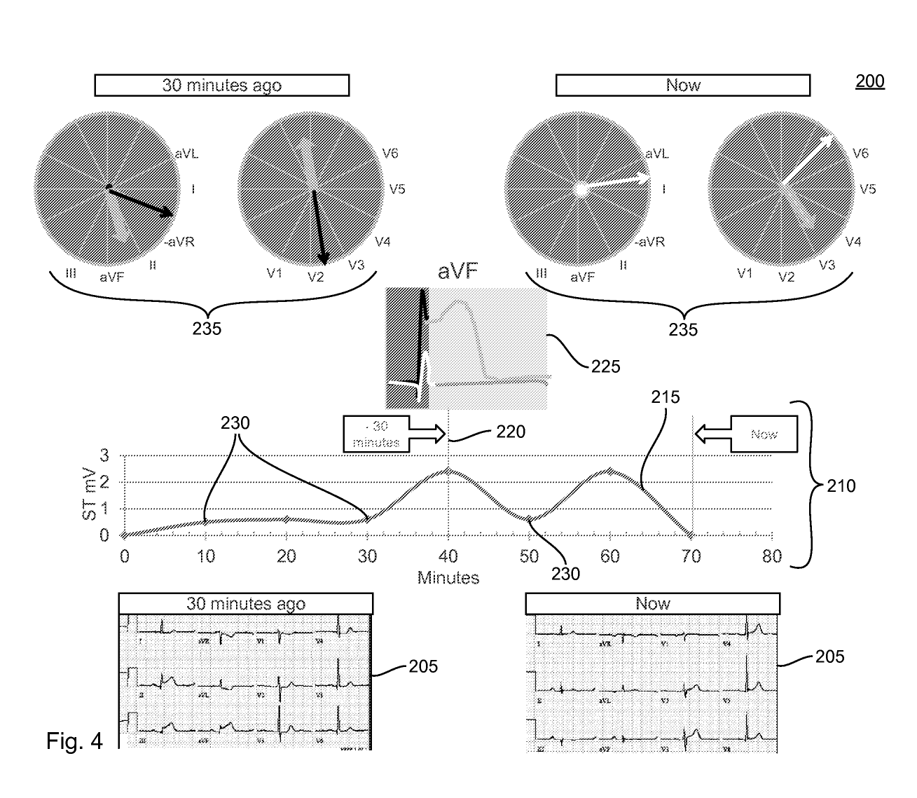

Once the method 100 is completed and the appropriate ECGs are selected, a second method 155 is carried out in order to construct an embodiment of the GUI 200 of the present application. Referring to FIG. 3 and FIG. 4, in the method 155, the trend 210 is rendered using the lead selected that reflects the greatest change. More specifically, rendering a curve 215 interpolated from actual points 230 along the trend 205 obtained from the 12-lead ECGs. The method 155 identifies two ECGs on the trend in step 165 that represent that greatest change. As illustrated in FIG. 4, this could he the current ECG versus one acquired minutes or hours ago. In step 170, the method 155 generates thumbnail views 205 of those ECGs that generated the specific sample point 220, and in step 175 overlays the cardiac complex 225 from the specific lead selected from each of these ECGs and displays it above the trend 210. Finally, in step 180, the method 155 identifies which portion, (i.e. time segment), of the cardiac complex 225 was used to generate the points 230 along the trend 210.

Still referring to FIG. 4, in order to navigate through the information provided in the GUI 200, the user must be able to move the sample point 220 that represents a time point for either ECG selected from the trend 210. In the FIG. 4 GUI 200, the exemplary examples are “now” and “30 minutes ago”. When the sample point 220 is moved by a user, the cardiac complexes 225 that have been superimposed over one another change to reflect the new selection of time along the trend 210 and the thumbnails 205 for those specific. ECGs where these cardiac complexes 225 are updated along the bottom of the trend 210. The user can also select any trend 210 they want to display that has been calculated by the system.

Since the trend 210 and the thumbnails 205 do not contain spatial information that was calculated from the 12-leads, another visual cue can be added to the GUI 200. In the example of FIG. 4, the spatial vectors 235 are displayed showing projection onto the horizontal or frontal plane of the 12-lead ECG.

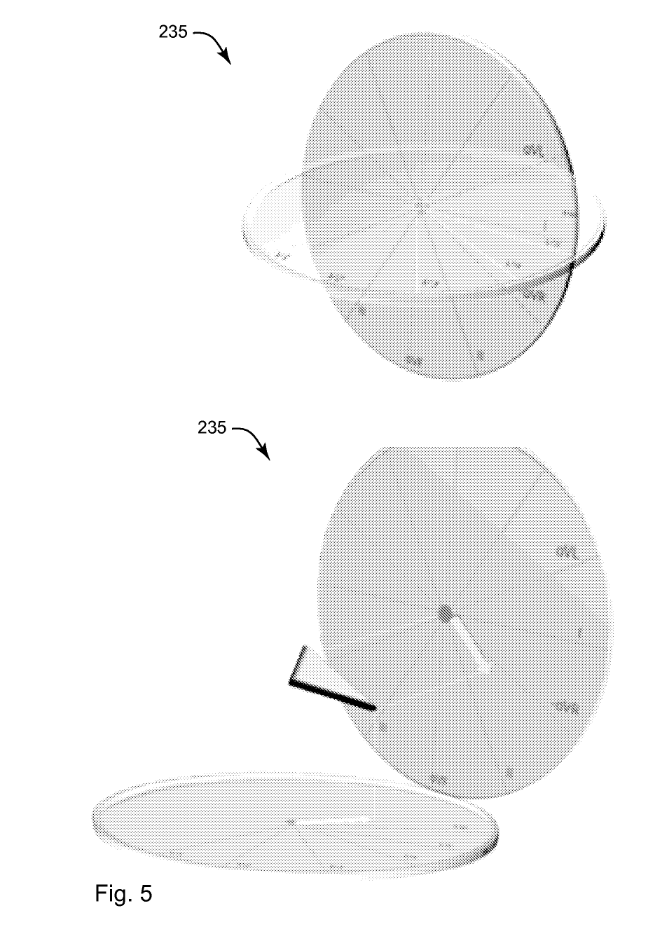

FIG. 5 simply illustrates how the spatial vectors 235 of the GUI 200 of FIG. 4 can be combined to construct a 3-dimensional (3D) version of the spatial vectors 235. This 3D version may be included in the GUI 200 in lieu of or in addition to the 2D versions.

In an additional embodiment, instead of using a single spatial vector 235 from one segment of the cardiac complex 225, two vectors 235 may be calculated for different segments of the cardiac complex 225 and compared. An example would be the spatial angle between the QRS complex and T-wave or ST T-wave.

In a further embodiment, instead of the comparison of two vectors 235, a feature value or index, in other words, a combination of measurements may be extracted from the representative complex 225 for each lead of the 12-lead ECG. The intent of these feature values would be to quantify those visual aspects of the ECG that reflect disease. An example of such a feature value is the ratio of the ST amplitude to the T-wave amplitude. Another may be the spatial angle between the T and ST wave. All the trends 210 may be analyzed according to the method 100 to find those that show the most marked change. Then a specific lead may be selected and the commensurate feature value trended.

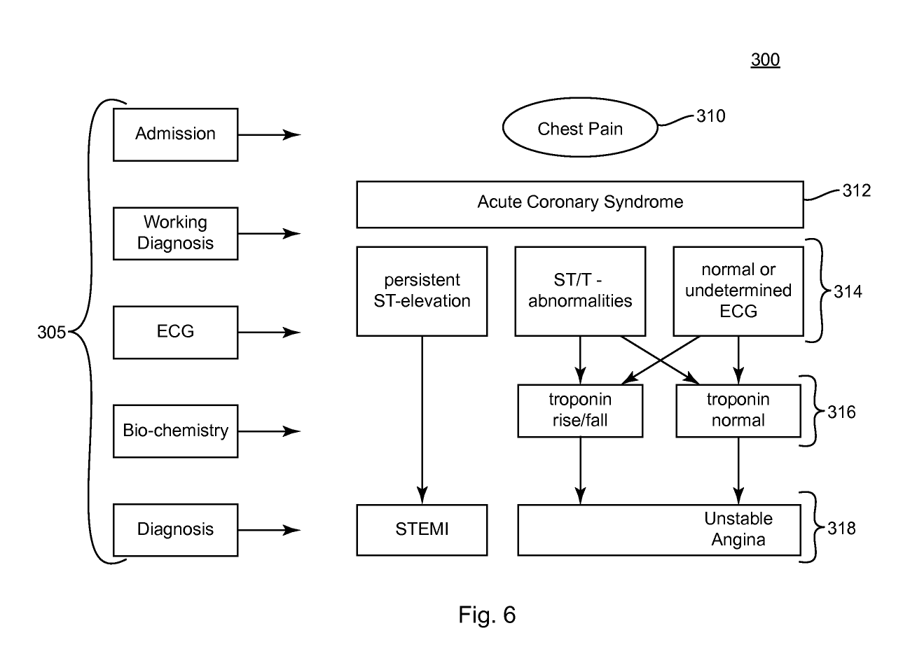

Referring now to FIG. 6, an additional method 300 of the present application is illustrated. This method 300 includes and illustrates the various patient stages 305 from an admission of the patient to a medical facility for treatment through final diagnosis. The previous described methods 100, 155, may be utilized within this method 300, specifically to derive the ECG results 314, which will be discussed below. The patient stages 305 first include an admission symptom 310. In other words, when a patient is admitted to an emergency room or other medical facility visit that requires an ECG, an initial diagnosis will be made based upon the symptoms the patient is currently experiencing. A working diagnosis 312 is then made based on the admission symptoms 310 reported by the patient. The working diagnosis 312, and in this exemplary case an ACS, is made by a medical professional. The ECG results 314, in this exemplary case, persistent ST elevation, ST/T abnormalities, and/or normal or undetermined ECG are made utilizing the methods 100, 155 described previously in this application. In step 316, biochem issues are factored in, in other words, how certain anesthetics and/or other medicaments that are known to be in the user's system may affect the ECG results 314. Finally, a final diagnosis 318 is made based upon all of the previous steps.

Still referring to FIG. 6, the number of ECG results 314 that may be obtained include all of the abnormalities discussed previously and various others known in the art.

When the system and method is asked to assess a patient suspected of an ACS, the following will be identified by the program;
Malignant arrhythmias or high-degree AV block:
Changes in conduction indicative of an acute coronary syndrome;
Changes in conduction that can result in a secondary repolarization abnormality;
Changes in repolarization (ST/T & QT) associated with unstable angina/NSTEMI;
Changes in repolarization (ST/T/QT) which make it clearer that the patient is suffering from a STEW:
Small changes in Q-waves; and
In addition to the use of conventional ECG measurements, the system and method may trend the probability of acute ischemia based on advanced algorithms.

Furthermore, the step of evaluating the bio-chem issues 316 may likewise detect and/or analyze a number of different bio-chem issues.

When the system and method is asked to evaluate a patient who has been administered a drug known to prolong QT and cause sudden cardiac death, the following will be identified:
Changes in conduction that disallow an evaluation of ST/T;
Changes in the ECG that increase the risk of sudden death due to prolonged QT, such as abnormal T wave morphology, bradyarrhythmias, PVC's with a short-long-short sequence of RR intervals, T-wave alternans, and abnormal U waves, etc.; and
Changes in the T-wave that are known to track plasma levels of drugs known for causing sudden cardiac death.

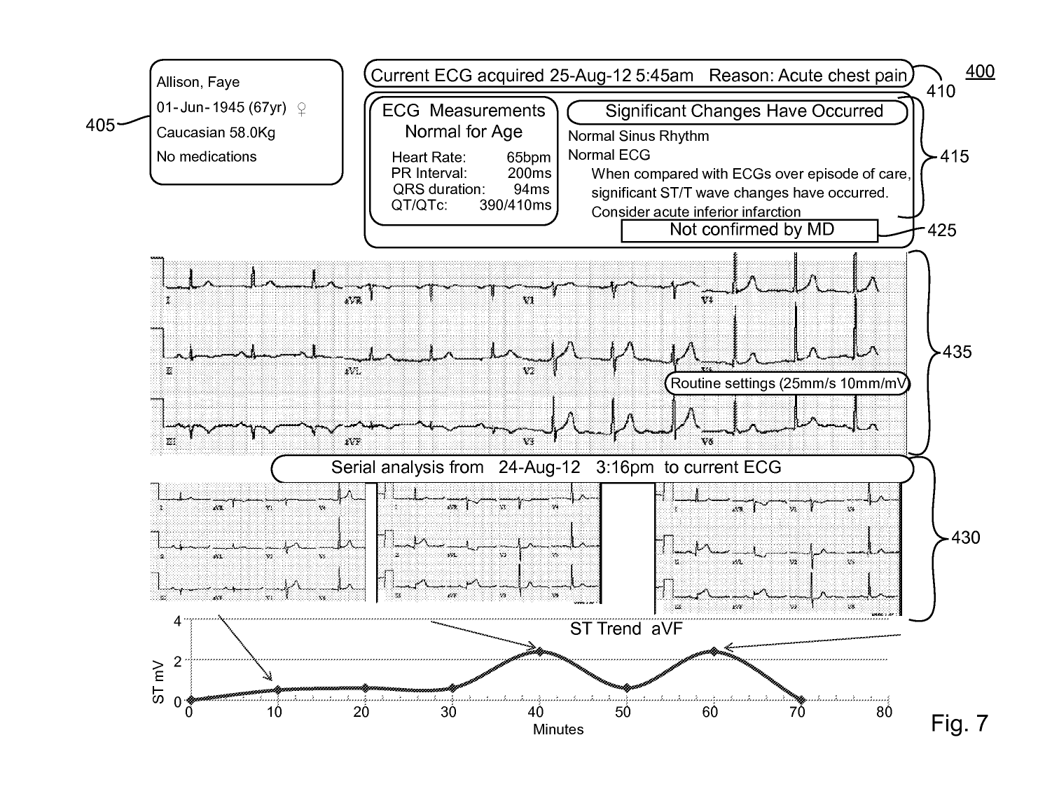

As discussed previously, the final diagnosis 318 may be derived and displayed for the user to analyze according to the GUI and FIG. 7.

Referring now to FIG. 7, an additional embodiment of a graphical user interface (GUI) of the present application is illustrated. Here, a patient information section 405 provides the user with the general information for a patient being monitored, including but not limited to, the patient's name, birthdate, age, sex, race, weight and whether that patient is currently medicated. The status bar 410 provides the user with information regarding when the ECG was acquired and the admission symptoms that prompted the ECG. The ECG change status 14 illustrates first whether any significant changes have occurred in the serial ECG analysis, and the current ECG status 420 illustrates for the user the status of the current ECG and whether that ECG is normal or abnormal. The serial ECG 430 and analysis illustrate the current ECG as well as select ECGs and trends as described previously with respect to FIGS. 4 and 5. Last, serial ECG waveforms 435 are also illustrated for the user.

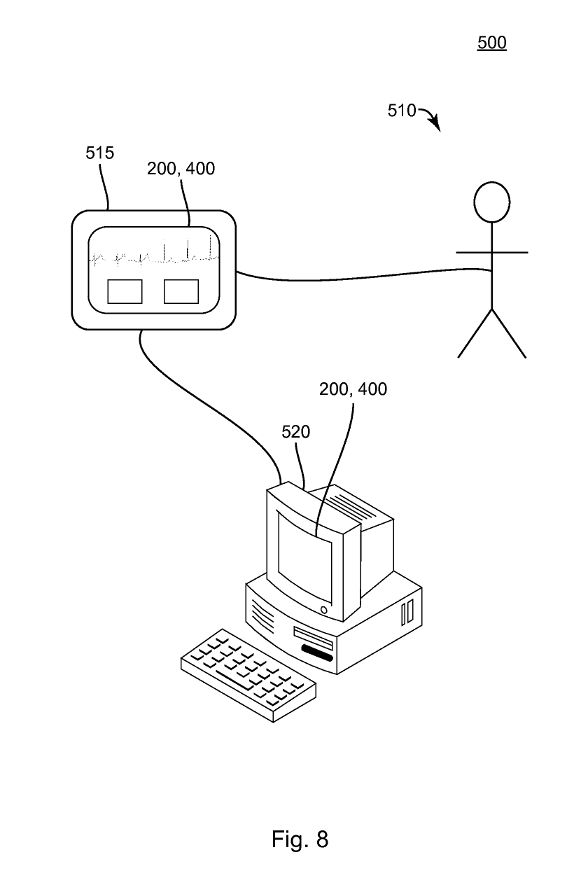

Referring now to FIG. 8, a system 500 of the present application is illustrated. Here, a patient 510 is being, monitored by an ECG monitor 515. It should first be noted that the ECG monitor 515 can be any monitor known in the art, portable, stationary or otherwise, that is utilized by physicians and medical professionals to monitor an ECG of a patient 510. The ECG monitor 515 is configured to provide the user with ECG waveforms for the patient 510 and also any of the graphical user interfaces 200, 400 as described above. In one embodiment, a user may monitor the patient 510 using the ECG monitor 515 and graphical user interfaces 200, 400 and all the functionality as described above, or alternatively, a separate computing device 520 may be utilized to retrieve ECG data from a patient 510 from the ECG monitor 515 in order to analyze the data according to the present application and utilize the graphical user interfaces 200/400.

The ECG monitor and/or computing device 520 also includes a controller (not shown). The controller includes a processor, storage system, software, and a communication interface. The controller may load and execute software from the storage system, including a software module. When executed by the controller, the software directs the processor to operate as described herein in further detail. Specifically, the controller may control the operation of all of the methods and resulting GUIs described previously in this application.

Although the controller as described above includes one software module in the present example, it should be understood that one or more modules could provide the same operation. Similarly, while the description as provided herein refers to a controller and a processor, it is to be recognized that implementations of such systems can be performed using one or more processors, which may be communicatively connected, and such implementations are considered to be within the scope of the description.

The processor can comprise a microprocessor and other circuitry that retrieves and executes software from storage system. The processor can be implemented within a single processing device but can also be distributed across multiple processing devices or sub-systems that cooperate in executing program instructions. Examples of processors include general-purpose central processing units, application specific processors, and logic devices, as well as any other type of processing device, combinations of processing devices, or variations thereof.

The storage system can comprise any storage medium readable by processor, and capable of storing software. The storage system can include volatile and nonvolatile, removable and non-removable media implemented in any method or technology for storage of information, such as computer readable instructions, data structures, program modules, or other data. Storage system can be implemented as a single storage device but may also be implemented across multiple storage devices or subsystems. Storage system can further include additional elements, such as a controller capable of communicating with the processor.

Examples of storage media include a random access memory, read only memory, magnetic disks, optical disks, flash memory, virtual memory, and non-virtual memory, magnetic sets, magnetic tape, magnetic disk storage or other magnetic storage devices, or any other medium which can be used to store the desired information and that may be accessed by an instruction execution system, as well as any combination or variation thereof, or any other type of storage medium.

Any of the ECG monitor, computing system or other ECG device will have a user interface that can include a mouse, a keyboard, a voice input device, a touch input device for receiving a gesture from a user, a motion input device for detecting non-touch gestures and other motions by a user, and other comparable input devices and associated processing elements capable of receiving user input from a user. Output devices such as a video display or graphical display can display and interface further associated with embodiments of the system and method as disclosed herein. The speakers, printers, haptic devices, and other types of output devices may also be included in the user interface.

As described above, the system and method is configured to improve the operation of current systems and computers implementing those systems such that changes between two ECGs may not be as important as an overall trend of changes across many ECGs, and the serial comparison program will not necessarily be tied to an ECG management system. Rather, it may be portable and able to execute in the device acquiring multiple ECGs from a single patient, such as a bedside monitor. Furthermore, the serial comparison program will be able to refrain from using a specific ECG due to a lack of ECG quality, and will generate a summary report for the episode-of-care, which will include at least one trend of a specific ECG measurement. Also, the system and method may be told what it is to be used for, such as evaluation of a patient suspected of an acute coronary syndrome or as the result of the administration of a drug known to prolong QT and cause sudden cardiac death. This allows the system and method to focus on key aspects related to the problem, rather than what is done in the heart station where a prior ECG is consulted under most circumstances, and will take into account time-stamped, non-ECG information supplied during the episode-of-care, including: a change in patient symptoms, drugs administered, procedures done, changes of body position, re-application of ECG leads, and lab results

In the foregoing description, certain terms have been used for brevity, clearness, and understanding. No unnecessary limitations are to be inferred therefrom beyond the requirement of the prior art because such terms are used for descriptive purposes and are intended to be broadly construed. The different configurations, systems, and method steps described herein may be used alone or in combination with other configurations, systems and method steps. It is to be expected that various equivalents, alternatives and modifications are possible within the scope of the appended claims.

1. A method of serial comparison of a plurality of 12-lead electrocardiograms (ECGs) during an episode of care, comprising:
determining a vector for each of the plurality of 12-lead ECGs, wherein each vector represents energy for a time segment;
generating a trend for each vector;
calculating a signal quality for a set of candidate ECGs selected from the plurality of 12-lead ECGs; and
determining an ECG from the set of candidate ECGs that best reflects as change in the trend.

2. The method of claim 1, wherein the determining step further includes synthesizing a set of XYZ components for each of the plurality of 12-lead ECGs.

3. The method of claim 2, wherein the determining step further includes computing a representative complex for each of the plurality of 12-lead ECGs and the set of XYZ components.

4. The method of claim 3, wherein the determining step further includes integrating a voltage for each of the X, Y and Z components, and further calculating a vector magnitude and a spatial angle for each vector.

5. The method of claim 1, further comprising calculating a signal quality for an additional set of ECGs in close time proximity to the set of candidate ECGs.

6. The method of claim 1, further comprising rendering the trend using a lead selected that reflects a greatest change.

7. The method of claim 1, further comprising identifying a pair of ECGs on the trend that represents the greatest change.

8. The method of claim 1, further comprising generating a set of thumbnail views of the pair of ECGs.

9. The method of claim 1, further comprising overlaying a cardiac complex from a selected lead from each of the plurality of ECGs in the trend.

10. The method of claim 1, further comprising identifying a portion of the cardiac conic ex that is used to generate the values along the trend.

11. A system of serial comparison of a plurality of 12-lead electrocardiograms (ECGs) during an episode of care, comprising:
a controller configured to determine a vector for each of the plurality of 12-lead ECGs, wherein each vector represents energy for a time segment, and generate a trend for each vector, the controller further configured to calculate a signal quality for a set of candidate ECGs selected from the plurality of 12-lead ECGs, and to determine an ECG from the set of candidate. ECGs that best reflects a change in the trend; and
a graphical user interface configured to display the trend using a lead selected that reflects a greatest change.

12. The system of claim 11, wherein the controller synthesizes a set of XYZ components for each of the plurality of 12-lead ECGs,

13. The system of claim 12, wherein the controller computes a representative complex for each of the plurality of 12-lead ECGs and the set of XYZ components.

14. The system of claim 13, wherein the controller integrates a voltage for each of the X, Y and Z components, and further calculates a vector magnitude and a spatial angle for each vector.

15. The system of claim 11, wherein the controller calculates a signal quality for an additional set of ECGs in close time proximity to the set of candidate ECGs.

16. The system of claim 11, wherein the controller identifies a pair of ECGs on the trend that represents the greatest change.

17. The system of claim 11, wherein the controller generates and the graphical user interface displays a set of thumbnail views of the pair of ECGs.

18. The system of claim 11, wherein the graphical user interface overlays a cardiac complex from a selected lead from each of the plurality of ECGs in the trend.

19. The system of claim 11, wherein the controller identifies a portion of the cardiac complex that is used to generate the values along the trend.

20. A method of serial comparison of a plurality of 12-lead electrocardiograms (ECGs) during an episode of care, comprising:
synthesizing a set of XYZ components for each of the plurality of 12-lead ECGs;
computing a representative complex for each of the plurality of 12-lead ECGs and the set of XYZ component;
integrating a voltage for each of the X, Y and Z components, and further calculating a vector magnitude and a spatial angle for each vector magnitude;
generating a trend for each vector magnitude;
calculating a signal quality for a set of candidate ECGs selected from the plurality of 12-lead ECGs;
determining an ECG from the set of candidate ECGs that best reflects a change in the trend; and
rendering the trend using a lead selected that reflects a greatest change.
